(19) United States
(12) Patent Application Publication
(10) Pub. No.: US 2018/0169319 A1
(43) Pub. Date: Jun. 21, 2018

(54) Treatment of cancer by manipulating the immune system

(71) Applicant: Marv Enterprises, LLC, Hermitage, PA (US)

(72) Inventor: Mitchell S. Felder, Hermitage, PA (US)

(21) Appl. No.: 15/786,883

(22) Filed: Oct. 18, 2017

Related U.S. Application Data
(63) Continuation of application No. 14/390,182, filed on Oct. 2, 2014, now abandoned.
(60) Provisional application No. 61/649,892, filed on May 21, 2012.

(51) Int. Cl. A61M 1/36; C07K 16/30; C07K 16/22; C07K 16/00; C07K 16/18

(52) U.S. Cl. CPC A61M 1/362; C07K 16/30; C07K 16/22; C07K 16/18; C07K 2319/31; C07K 16/00

(57) ABSTRACT

The present invention relates to the treatment of cancers. Specifically, the invention pertains to a method for the extracorporeal treatment of one or more body fluids (e.g., blood or cerebrospinal fluid, CSF) in two stages characterized by removing a body fluid (e.g., blood or CSF) from a living body diseased with a type of cancer, passing the body fluid through a first stage of applying a treatment to at least one antigen in the body fluid. More specifically, the treatment comprises creating an antibody-antigen moiety during passage thereof through said first stage, passing the treated body fluid through a second stage, removing antibody-antigen moiety from the body fluid during passage through the second stage, and returning the purified body fluid to the body.

### CROSS-REFERENCE TO RELATED APPLICATIONS

This application claims benefit under 35 U.S.C. § 119(c) of U.S. Patent Application No. 61/649,892, filed May 21, 2012, which is hereby incorporated herein by reference in its entirety.

### BACKGROUND OF THE INVENTION

In the United States cancer is the second leading cause of death. Cancer has surpassed heart disease is the number one cause of death in patients younger than 85 years of age. In the United States each year 1.3 million cases of cancer are diagnosed and more than 570,000 people die each year from cancer. The highest mortality rates involving lung, colorectal, breast and prostate cancers. More people presently die from cancer in the United States each year than were killed in all of the wars in which the United States participated during the twentieth century. The average age at the time of diagnosis for cancer is 67 years, and about three quarters of all cancers are diagnosed after the age of 55. Further, cancer is the second leading cause of death in children up to the age of fourteen. In children leukemia is the most common cause of death from cancer. There are over 8 million Americans alive today who have had some form of cancer. Approximately one in three Americans will develop some form of cancer within their lifetime.

Certain molecular organic compounds are implicated in inducing a release of an immune systemic response which causes a remission of cancerous tumors and metastases.

### SUMMARY OF THE INVENTION

In general terms, the present invention relates to the treatment of cancers, hereinafter abbreviated as “CA”. Specifically, the invention pertains to a method for the extracorporeal treatment of one or more body fluids (blood or CSF: cerebrospinal fluid) in two stages characterized by removing a body fluid (blood or CSF: cerebrospinal fluid) from a living body diseased with a type of CA, passing the body fluid through a first stage; applying a treatment to at least one antigen in the body fluid. The mechanism of action of this treatment methodology is to increase the activity of T-cells by removing those proteins which are T-cell inhibitors. This thereby concomitantly breaks down the intrinsic tolerance to tumor-associated antigens in the host patient. By utilizing a finely tuned concentration, via this extra-corporeal methodology of target antigen(s) removal, there is the avoidance of autoimmune reactions against self-tissue antigens within the host patient. This avoids concomitant endocrinopathies, hepatitis, dermatitis and enterocolitis during the treatment methodology.

More specifically, the treatment comprises creating an antibody-antigen moiety during passage thereof through said first stage; passing the treated body fluid through a second stage; removing antibody-antigen moiety from the body fluid during passage through the second stage, and returning the purified body fluid to the body.

The invention is further characterized by targeting an antigen in the body fluid, with an antibody to allow and facilitate removal thereof in the second stage. The targeted antigen(s)/TA(s) would include one, or a combination of: PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1).

Specifically, the method is further characterized by removing body fluid from a person to produce the extracorporeal bodily fluid; imposing a treatment acting on an antigen of CA in the body fluid, filtering or otherwise removing the treatment from the body fluid, and returning the body fluid to the patient after removing substantially all of the treatment in the second stage.

The method of the present invention comprises treating at least one component of a patient's body fluid extracorporeally with a designer antibody containing an albumin-moiety which will create an albumin-antibody-antigen moiety allowing for the efficacious dialysis of the resultant albumin-antibody-antigen compound (the targeted antigen being respectively, one or a combination of target antigen(s)/TA(s) from: PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1).

More specifically, the method is characterized by removing body fluid from a person to produce the extracorporeal bodily fluid; directing a first antibody against the targeted antigen(s)/TA(s): PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1), in the first stage of extra-corporeal treatment in the body fluid; in the second stage directing a second antibody conjugated with albumin and/or a protein against the targeted antigen(s) thereby forming an albumin-antibody-antigen compound; removing at least a substantial portion of the albumin-antibody-antigen compound from the body fluid by dialysis, other filtering, or other means; and returning the body fluid to the patient.

Also, the method is characterized by testing the blood and/or CSF to determine the efficacy of treatment before returning the body fluid to the patient. U.S. Ser. No. 13/128,870, U.S. Ser. No. 13/128,177, U.S. Ser. No. 13/254,855, U.S. 61/612,474 and U.S. 61/644,292 are hereby incorporated by reference.

### BRIEF DESCRIPTION OF THE DRAWING

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

[12] In the first stage of treatment, a selected body fluid is removed using a standard catheter and/or lumbar puncture. In the second stage, the body fluid is treated with antibodies against the targeted antigen(s) TA(s): PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1).

The method of the present invention comprises treating at least one component of a patient's body fluid extracorporeally with a designer antibody containing an albumin-moiety to create an albumin-antibody-antigen moiety allowing for the efficacious dialysis, filtering or other means of removal of the resultant albumin-antibody-antigen compound (the targeted antigen being respectively, one or a combination of antigen(s) from: PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1),

The albumin-antibody will be directed towards facilitating removal of the targeted antigen(s)/TA(s) : PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1).

After the removal of one or more of the target antigen(s)/TA(s) the cleansed body fluid will be returned to the patient. The frequency of treatment, and the specifically targeted antigen(s) to be removed would depend upon the underlying symptomatology and pathology of the patient, and would be determined by the patient's physician. The article used in performing the method includes two-stages. The first stage includes a treatment chamber for addition of an antibody with an attached albumin moiety, which is added to the body fluid. A second stage receives the treated blood and/or CSF and includes a unit for removing the treatment.

The method includes providing a dialysis or other filtering machine with a first stage and a second stage, and sequentially passing the extracorporeal body fluid through the first and second stages. The body fluid is removed from the patient using standard procedure. The first stage applies a treatment using an antibody which was has attached to it an albumin moiety (or alternatively, a moiety which allows for the efficacious dialysis or removal by other techniques of the antibody-albumin-CA antigen), for the treatment of the body fluid. The second stage substantially removes the treatment. The purified body fluid (body fluid with removed targeted antigen(s)/TA(s): PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1),—is then tested for the efficacy of removal of the antigen and returned to the patient.

An alternative methodology of the present intervention would utilize a designer antibody with an attached macromolecular moiety instead of an albumin moiety. The macromolecular moiety, attached to the antibody, would be 1.000 mm to 0.005 mm in diameter. The antibody-macromolecular moiety-targeted antigen complex would then be blocked from reentering the patient's body fluid circulation, by utilizing a series of microscreens which contain openings with a diameter 50% to 99.9999% less than the diameter of the designer antibody-macromolecular moiety. The microscreen opening(s) must have a diameter of at least 25 micrometers in order to allow for the passage and return to circulation of the non-pathologic inducing body fluid constituents.

Alternatively, the target antigens (TAs): PD-1 (Programmed Death 1), CTLA-4 (Cytotoxic T-Lymphocyte Antigen 4; or CD152: Cluster of Differentiation 152), PP14 (Placental Protein 14), TGF-Beta 1 (Transforming growth factor beta 1), may be captured by utilizing antibody microarrays which contain antibodies to targeted antigens. The antibody microarrays comprise millions of identical monoclonal antibodies attached at high density on glass or plastic slides. After sufficient extracorporeal exposure of the targeted antigens to the antibody microarrays, the antibody microarrays-targeted antigens may be disposed of utilizing standard medical practice.

Another alternative methodology of the present intervention comprises removing one or more of the targeted cancer antigens from the body fluid by utilizing a designer antibody containing an iron (Fe) moiety. This will then create an Fe-Antibody-Antigen complex. This iron containing complex may then be efficaciously removed utilizing a strong, localized magnetic force field. Alternatively, immunoaffinity chromatography may be employed in which the heterogeneous group of molecules in the body fluid will undergo a purification process. There will be an entrapment on a solid or stationary phase or medium. Only the targeted antigens (TAs) will be trapped utilizing immunoaffinity chromatography. A solid medium can be removed from the mixture, washed, and the TA(s) may then be released from the entrapment through elution.

Alternatively, gel filtration chromatography may be utilized in which the body fluid is used to transport the sample through a size exclusion column that will be used to separate the target antigen(s)/TA(s) by size and molecular weight.

Another alternative methodology of the present intervention would utilize a molecular weight cut-off filtration. Molecular weight cut off filtration refers to the molecular weight at which at least 80% of the target antigen(s)/TA(s) are prohibited from membrane diffusion.

The target antigen(s) can be differentiated utilizing standard ELISA methodology. ELISA (enzyme-linked immunosorbant assay) is a biochemical technique which allows for the detection of an antigen in a sample. In ELISA an antigen is affixed to a surface, and then an antibody is utilized for binding to the antigen. The antibody is linked to an enzyme which enables a color change in the substrate. Other strategies may be employed to validate the level of target antigen(s)/TA(s) in the body fluid: Western blotting technology, UV/Vis spectroscopy, mass spectrometry, and surface plasmon resonance (SPR).

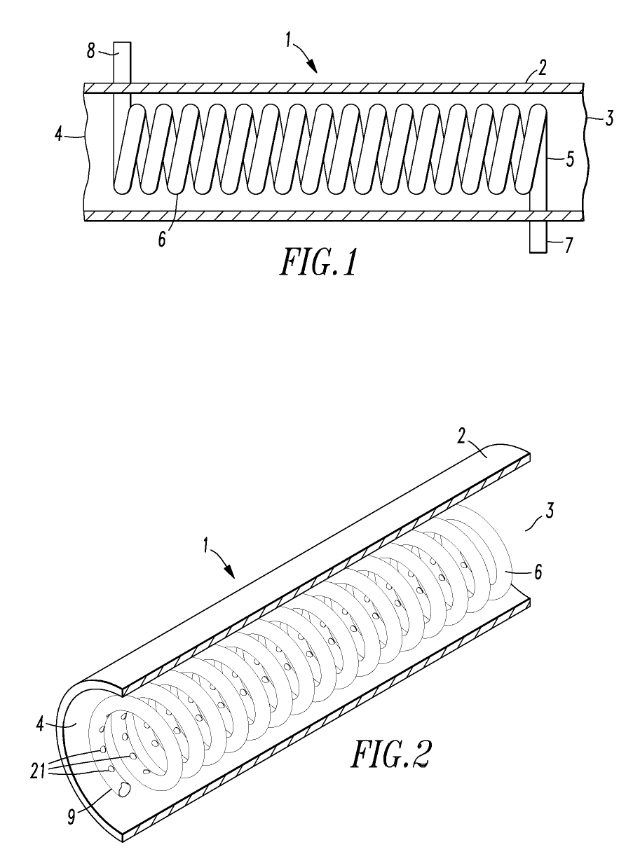

The device of the invention includes a first stage and a second stage. The first stage applies a treatment of an antibody with an attached albumin moiety targeting the antigen(s) specifically exacerbating the pathologic condition. The second stage includes substantial removal of the treatment from the extracorporeal body fluid bodily fluid. As shown in FIG. 1, the first stage can include an exterior wall to define a treatment chamber 5. The treatment conveniently can be applied in the treatment chamber 5. Residence times of the body fluid can be altered by changing the dimensions of the treatment chamber, or by using a dialysis vacuum pump. With reference to FIG. 1, body fluid enters the inlet 3, passes through the treatment chamber 5, and exits the outlet 4. In embodiments, the treatment of an antibody with an attached albumin moiety targeting the CA antigen(s) can be applied from a delivery tube 6 located within the treatment chamber 5. An inferior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the blood and/or CSF. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer anti bodies in the body fluid. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic. The antibody with attached albumin moiety, targeting the CA antigen(s) can be delivered in a concurrent or counter-current mode with reference to the body fluid. In counter-current mode, the body fluid enters the treatment chamber 5 at the inlet 3. The designer antibody can enter through a first lead 8 near the outlet 4 of the treatment chamber 5. The blood and/or CSF then passes to the outlet 4 and the designer antibodies pass to the second lead 7 near the inlet 3. The removal module of the second stage substantially removes the designer antibodies-CA antigen molecular compound from the body fluid.

The second stage can include a filter, such as a dialysis machine, which is known to one skilled in the art. The second stage can include a molecular filter. For example, molecular adsorbents recirculating system (MARS), which may be compatible and/or synergistic with dialysis equipment. MARS technology can be used to remove small to average sized molecules from the body fluid. Artificial liver filtration presently uses this technique.

The method can include a plurality of steps for removing the targeted antigen(s). A first step can include directing a first antibody against the targeted antigen. A second step can include a second antibody. The second antibody can be conjugated with albumin, or alternatively another moiety which allows for efficacious dialysis or filtering of the antibody-antigen from the body fluid. The second antibody or antibody-albumen complex combines with the first antibody forming an antibody-antibody-moiety complex. A third step is then used to remove the complex from the blood and/or CSF. This removal is enabled by using dialysis and/or MARS. The purified body fluid is then returned to the patient.

In practice, a portion of the purified body fluid can be tested to ensure a sufficient portion of the targeted antigen(s) have been successfully removed from the body fluid. Testing can determine the length of treatment and evaluate the efficacy of the sequential dialysis methodology in removing the targeted antigen(s) and suggest the need for further treatment. body fluid with an unacceptably large concentration of complex remaining can then be retreated and refiltered before returning the body fluid to the patient.

In embodiments, the second stage to remove the antibody-moiety-targeted CA antigen complex from the body fluid can be accomplished by various techniques including, for example, dialysis, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the antigen(s).

Further techniques can include continuous renal replacement therapy (CRRT) which can remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis. The sieving coefficient (SC) is the ratio of the molecular concentration in the filtrate to the incoming blood and/or CSF. A SC close to zero implies that the moiety-antibody-targeted antigen complex will not pass through the filter. A filtration rate of 50 ml per minute is generally satisfactory. Other methods of increasing the removability of the antibody-targeted antigen moiety include the use of temporary acidification of the body fluid extracorporeally using organic acids to compete with protein binding sites.

The invention can also be used in combination with other therapies including, for example, Kanzius radiofrequency (RF) therapy as described in U.S. Pat. No. 7,510,555 and U.S. Pat. No. 7,627,381 which are hereby incorporated by reference. Kanzius therapy presently uses nanoparticles and RF radiation to induce hyperthermia in cancer cells. In this methodology, the Kanzius methodology would be utilized to break down the molecular structure of the targeted antigen(s)/TA(s).

The invention and Kanzius therapy are synergistic. Alone, Kanzius therapy can cause multiple infarctions in major organs leading to blindness, heart attacks, and renal failure.

Performing Kanzius therapy extracorporeally avoids these morbidities. Additionally, much higher levels of RF can be used. The invention can include a treatment comprising Kanzius therapy. Nanoparticle residue of the Kanzius therapy and cellular and/or pathogen debris can be substantially removed from the blood in the second stage. Reducing the residue and debris returned to a patient's vascular system can reduce deleterious vascular cascades such as coagulation and inflammation, which are further causes of patient morbidity.

Advantageously, a physician can use magnetic resonance angiography (MRA) or magnetic resonance venography (MRV) to determine the arterial and venous blood vessels to and from a tumor. These techniques can identify the blood vessels from which the extracorporeal blood can be extracted and into which the treated blood can be returned.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for extracorporeal treatment of body fluid in two stages comprising:
a. removing a body fluid from a patient;
b. applying a treatment to at least one target antigen in the body fluid to create an antibody-antigen moiety; and
c. removing the antibody-antigen moiety from the body fluid before returning the body fluid to the patient.

2. The method of claim 1, wherein the target antigen is selected from the group consisting of PD-1 (Programmed Death 1), CTLA-4 (Cytotoxic T-Lymphocyte Antigen 4; or CD152: Cluster of Differentiation 152), PP14 (Placental Protein 14), TGF-beta 1 (Transforming growth factor beta 1), and combinations thereof.

3. The method of claim 1, wherein the treatment comprises:
a. directing a first antibody against the targeted antigen; and
b. directing a second antibody conjugated with a protein against the targeted antigen thereby forming an protein-antibody-antigen compound; and
c. removing at least a portion of the albumin-antibody-antigen compound from the body fluid.

4. The method of claim 3, wherein the protein comprises albumin.

5. The method of claim 1, wherein the body fluid is selected from a group consisting of blood, cerebral spinal fluid, and lymph.

6. The method of claim 1 further including testing the body fluid for efficacy before returning the body fluid to the patient.
